(19) United States
(12) Patent Application Publication
(10) Pub. No.: US 2013/0296646 A1
(43) Pub. Date: Nov. 7, 2013

(54) NON-INVASIVE OR MINIMALLY INVASIVE PARASPINAL SYMPATHETIC ABLATION FOR THE TREATMENT OF RESISTANT HYPERTENSION

(71) Applicant: ENIGMA MEDICAL, INC., San Diego, CA (US)

(72) Inventors: Denise Barbut, New York, NY (US); Allan Rozenberg, San Diego, CA (US); Axel Heinimann, New York, NY (US)

(73) Assignee: Enigma Medical, Inc., San Diego, CA (US)

(21) Appl. No.: 13/787,375

(22) Filed: Mar. 6, 2013

Related U.S. Application Data
(60) Provisional application No. 61/641,599, filed on May 2, 2012.
(60) Provisional application No. 61/724,086, filed on Nov. 8, 2012.
(60) Provisional application No. 61/733,034, filed on Dec. 4, 2012.
(60) Provisional application No. 61/739,396, filed on Dec. 19, 2012.

(51) Int. Cl. A61B 18/14

(52) U.S. Cl. CPC A61B 18/1492
USPC 600/104; 604/500; 606/33; 606/14; 606/41; 606/20; 604/187

(57) ABSTRACT

A method of ablating the sympathetic ganglionic cell bodies in the thoracic paravertebral space is provided. The method includes ablating the sympathetic ganglionic cell bodies in the thoracic paravertebral space through a posterior, non-invasive or minimally invasive approach for the treatment of resistant hypertension. The ablation may additionally involve various permutations of the gray and white rami and the dorsal root ganglion in addition to the sympathetic chain ganglionic cell bodies, all located in the triangular paravertebral space.

This application claims priority to U.S. Ser. No.: 61/641,599, filed on May 2, 2012, and U.S. Ser. No.: 61/724,086, filed on Nov. 8, 2012, and U.S. Ser. No.: 61/733,034, filed on Dec. 4, 2012, and U.S. Ser. No.: 61/739,396, filed on Dec. 19, 2012, the entireties of which are incorporated herein by reference.

### FIELD OF THE INVENTION

The invention relates generally to the field of non-invasive or minimally invasive approaches for the treatment of resistant hypertension. In particular, the invention relates to ablating the paravertebral sympathetic ganglion cells in the thoracic paravertebral space through a posterior non-invasive or minimally invasive approach for the treatment of resistant hypertension.

### DESCRIPTION OF THE RELATED ART

Hypertension affects tens of millions of individuals. Untreated hypertension is associated with stroke, heart failure and renal failure. Most patients with hypertension are currently treated pharmacologically, many with multiple medications. A quarter of these patients are resistant to medication and their blood pressure poorly controlled, putting them at added risk for complications.

Activation of the sympathetic nervous system is thought to play a significant role in exacerbating hypertension in the later stages of the disease. Reducing such sympathetic activation has been shown to reduce blood pressure in these circumstances.

Recently, mechanical o ablation of the renal nerves surrounding the renal artery has been shown to reduce blood pressure in patients with resistant hypertension. The technique consists of an endovascular, arterial procedure and involves radiofrequency ablation of post-ganglionic renal nerve fibres, accessed through the wall of the renal arteries bilaterally. Renal artery denervation, as the procedure is known, has been shown to reduce systolic and diastolic pressures by up to 30 mm and 10 mm respectively, and to be persistent out to a year or more following the procedure. The incidence and severity of procedure related and late complications are as yet unknown, as is the long term benefit on blood pressure reduction. Renal nerve fibres regenerate, and the hypotensive effect of this ablative procedure may diminish over time.

Therefore, alternatives to these therapies are needed which provide more significant reductions in blood pressure, persist indefinitely and which are safer, simpler, and less time-consuming.

### BRIEF SUMMARY OF THE INVENTION

The invention includes a method of ablating the sympathetic ganglionic cell bodies in the thoracic paravertebral space through a posterior, non-invasive or minimally invasive approach for the treatment of resistant hypertension. The ablation may additionally involve various permutations of the gray and white rami and the dorsal root ganglion in addition to the sympathetic chain ganglionic cell bodies, all located in the triangular paravertebral space.

In one aspect of the invention a method for treating resistant hypertension includes applying a stimulation ultrasonic or electric field to the paravertebral ganglion cell bodies and optionally also part of the peripheral nervous system; monitoring physiologic response to the stimulation field; and applying an ablating ultrasonic thermal field or a denervating electric field to the nervous tissue.

In another aspect of the invention a method for treating hypertension includes localizing paravertebral ganglionic cell bodies within the paraspinal space and inhibiting neural transmission through the tissue rather than denervating the tissue.

Applying the field may be done non-invasively using modalities such as high or low frequency ultrasound. Preferably, it may be done minimally invasively, by percutaneously threading an ablation wire into the paravertebral triangle.

In another aspect of the invention a method comprising reducing blood pressure of a patient by percutaneously accessing para-vertebral sympathetic ganglia, dorsal root ganglia or both in provided in which the ganglia are irreversibly disabled.

In a further aspect of the invention a method is provided, the method including reducing blood pressure of a patient by accessing a para-vertebral triangle; and irreversibly disabling neural structures therewithin.

In a further aspect of the invention a method is provided, the method including treating heart failure, acute myocardial infarction, renal disease, or chronic renal failure by percutaneously accessing para-vertebral sympathetic ganglia, dorsal root ganglia or both; and irreversibly disabling said ganglia.

In a further aspect of the invention a method is provided including stimulating para-vertebral sympathetic ganglia, dorsal root ganglia or both of a patient; monitoring a physiologic response related to the stimulating; applying ablative means to the para-vertebral sympathetic ganglia, dorsal root ganglia or both; and reducing blood pressure of the patient.

In a further aspect of the invention a method is provided, the method including stimulating a para-vertebral triangle; monitoring a physiologic response related to the stimulating; and applying ablative means to said para-vertebral triangle.

In a further aspect of the invention, a device for reducing blood pressure is provided, the device including an elongate tubular member with a proximal and distal end, adapted for percutaneous insertion proximate or within the para-vertebral sympathetic ganglia or dorsal root ganglia.

While multiple embodiments, objects, features, and advantages are disclosed, still other embodiments of the invention will become apparent to those skilled in the art from the following detailed description taken together with the accompanying figures, the foregoing being illustrative and not restrictive.

### BRIEF DESCRIPTION OF THE DRAWINGS

FIG. 1 is a diagram showing the anatomical location of the para-spinal sympathetic chain with the ganglia laying close to the antero-lateral third of the vertebral bodies and dorsal root ganglia more superficial, infero-lateral to the facet joints.

FIG. 2 is a CT scan through lower thoracic spine showing the position of an ablation catheter lateral to the vertebral body (arrow).

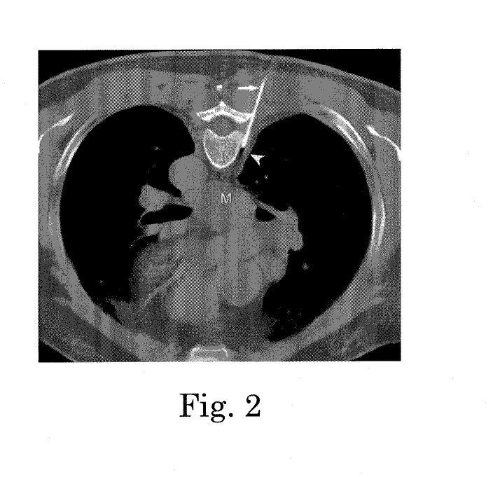

FIG. 3 is an illustration of a cross-section through the lower thoracic spine showing the position of the paravertebral triangle through which an ablation catheter is advanced.

FIGS. 4A and 4B are CT scans showing ablation of para-vertebral sympathetic ganglia using chemical means.

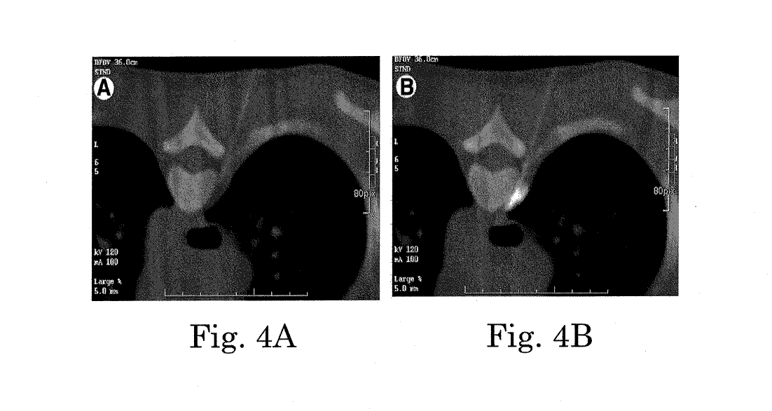

### DETAILED DESCRIPTION OF THE INVENTION

Hypertension is one of the most common chronic conditions I the world. It affects one in every 7 people globally, or 1 billion people. In the US alone, it affects 1 in 4 adults, close to 70M people. In Europe and Japan, the prevalence is almost double that in the US, affecting 50% or more of adults. It is a major risk factor for heart disease, congestive cardiac failure, stroke and renal failure. The total cost to society was nearly $80 billion in 2010. The risk of death doubles for every 20 mm increase in systolic blood pressure above 120 mm. Conversely, a 5 mm reduction in systolic pressure reduces the risk of stroke by 14%, the risk of heart disease by 9% and the overall mortality by 7%.

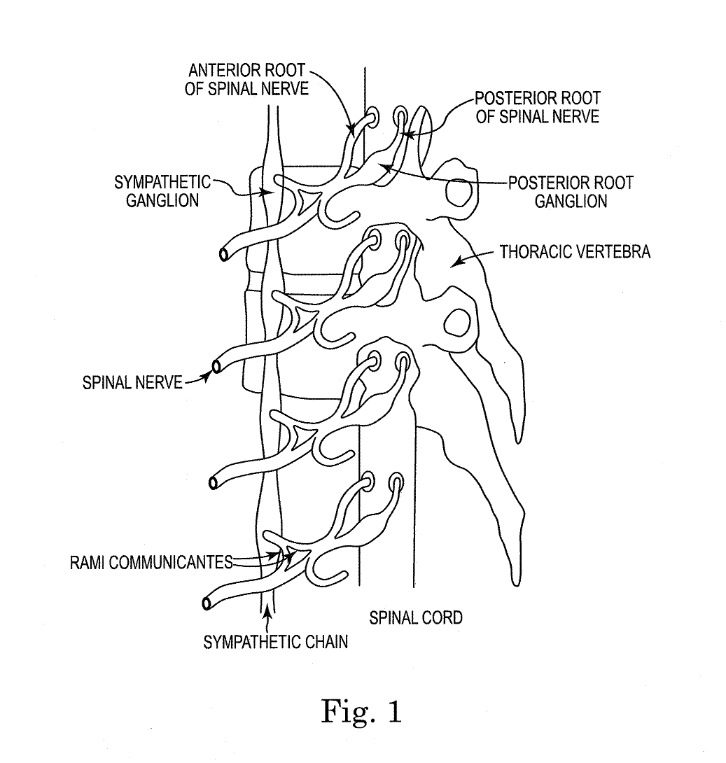

Afferent sympathetic nerve fibers from the kidney and the renal artery enter the spinal cord through the dorsal root ganglion. They ascend in the spinal cord to the autonomic control centers in the brain stem and brain. Efferent sympathetic fibers descend in the spinal cord and exit through the ventral root at each spinal level bilaterally. They traverse the white ramus communicantis and synapse with ganglionic cell bodies in the sympathetic paraspinal ganglia adjacent to the thoracic spine. From there, both pre-ganglionic and post-ganglionic axonscommunicate with neighboring paraspinal sympathetic ganglia or exit through the gray ramus communicantis to join the segmental spinal nerve. The segmental spinal nerves from T6 to L1, mostly from T8 to T11, first synapse on a pre-aortic ganglion cell and then ultimately reach the renal artery as best seen in FIG. 1.

The inventors propose denervating, inhibiting or ablating the sympathetic ganglion cells in the thoracic paravertebral space through a posterior non-invasive or minimally invasive approach for the treatment of resistant hypertension. The denervation, inhibition or ablation may also involve various permutations of the sympathetic ganglia alone, or in combination with the gray and white rami, the anterior nerve root, the spinal nerve and the dorsal root ganglion, all located in the triangular paravertebral space. This method of treating hypertension has not been previously described.

Surgical sympathetic denervation for the treatment of resistant hypertension was routinely performed in the 1940's. Such procedures involved removing various combinations of stellate ganglia in the neck, thoraco-lumbar paraspinal sympathetic ganglia, as well as splanchnic nerve excision. Blood pressure decreases were very significant, frequently associated with marked postural hypotension, and heart failure was improved. Such surgical procedures were also associated with significant procedural morbidity and mortality, and were rapidly abandoned in favor of pharmacologic treatments which became available in the 1950's. Pharmacotherapy became the mainstay of management for hypertensive patients during the second half of the last century. Many patients required more than one medication for adequate control of pressure, and up to a quarter of all remained hypertensive on multiple medications (resistant hypertension).

Recently, mechanical means of controlling blood pressure have been revisited, specifically for patients with resistant hypertension. Carotid sinus baroreceptor stimulation using implantable neurostimulation devices has been shown to reduce systolic pressures by up to 40 mm several years after the procedure. The only randomized clinical study using this device missed the primary shorter-term end-point however, and the study needs to be repeated. Furthermore, procedural complications attributable to the device were high. Renal artery denervation (RAD) involves ablating renal nerve fibres surrounding renal arteries bilaterally. The procedure involves advancing a catheter endovascularly into each of the renal arteries, and applying ablative energy through the wall of the artery to destroy some of the renal nerve fibres. The treatment lasts about 40 minutes. Procedure related complications are not uncommon. They include, transient bradycardia, embolization from atheromatous renal arteries to kidneys whose function may already be impaired by chronic hypertension, and renal artery spasm or dissection which may also cause deterioration in renal function. While both systolic and diastolic pressure improve following this treatment, the longer term effect on blood pressure is as yet unknown. Peripheral nerve fibres such as those within the renal nerve typically regenerate. Such regeneration following radiofrequency ablation has been demonstrated. Once a significant portion of ablated fibres regenerate, the beneficial effect of the procedure on blood pressure may be lost.

In this invention, we teach that denervation of paravertebral ganglion cell bodies rather than renal nerve fibres may be a more effective method of treating hypertension. Furthermore, there may be fewer complications associated with the procedure. The concentration of ganglion cell bodies within the paravertebral ganglion is very large. Hence ablating a small area will include a large proportion of the efferent signals to the kidney, whereas circumferentially ablating the renal artery is likely to include only a small proportion of nerve fibres. Furthermore, ablated ganglion cells don't regenerate, whereas renal nerve fibres can regenerate. Any reduction of blood pressure attributable to paravertebral ganglion cell ablation is thus likely to be permanent.

Surgical section of the dorsal root alone for pain control, has anecdotally been shown to prevent the development of hypertension in rodent models.

Lumbar radiofrequency ablation of the dorsal root is an established technique for the treatment of lumbar pain, and thoracic paravertebral anesthesia has been used for analgesia, in lieu of general anesthesia, during a variety of procedures including cholecystectomy, inguinal hernia repair and more recently, umbilical hernia repair.

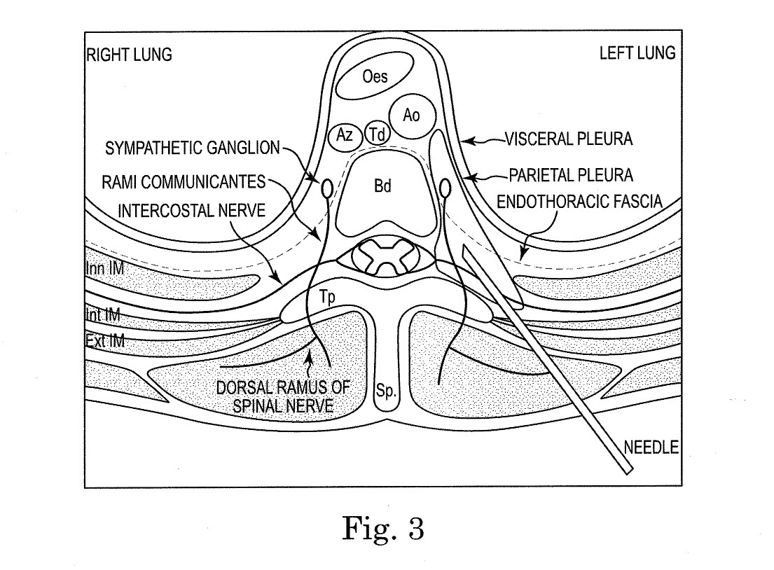

The thoracic paravertebral space (TPVS) is a triangular space delineated by the intervertebral discs, the vertebral body and the intervertebral foramina medially and the transverse process, the superior costo-transverse ligament and the ribs posteriorly. For the purposes of pain control, the dorsal root of the lumbar TPVS is easily accessed posteriorly using a 21 gauge needle and a nerve stimulator. The needle enters the paraspinal space lateral to the transverse process in the intervertebral space and is angled towards the spinous process. The sympathetic ganglia can be accessed by advancing the needle another 1.5-2 cm further anteriorly. The paravertebral sympathetic ganglia are apposed to the vertebral body antero-laterally. Within the posterior aspect of the TPVS, the initial stimulating current of 2.5 mA, 1 Hz, 9V typically causes contraction of the appropriate intercostals or abdominal muscle. The needle can then be cautiously advanced anteriorly until the appropriate muscle response can still be elicited but with a lower stimulating current of 0.1-0.5 mA. Once this has occurred, the stimulation parameters can be adjusted such that higher frequency stimulation inhibits ganglion cell firing. Assuming a lowering of blood pressure is detected, the ganglion is then ablated electrically using radiofrequency. The ablation may also be chemical, using sympatholytic agents such as phenol or capsaicin, or involve other methods such as , heat or cold, high or low frequency ultrasound, or any other method for inhibiting sympathetic transmission across the paravertebral sympathetic ganglion. Several procedures at several levels unilaterally or bilaterally may be required to achieve the desired level of blood pressure reduction. See FIGS. 1 and 3.

The inventive method is non-invasive or minimally invasive. It is performed by an anesthetist, neurosurgeon or neuroradiologist in an out-patient setting. The landmarks of the paraspinal TPVS can be identified ultrasonically or by CT or MRI quite easily. Small amounts of contrast can also be injected under radiographic control to determine the extent of communication between the TPVS.

The process may combine mapping with ablation in a sequential fashion. Procedures may initially be unilateral or bilateral and involve one thoracic level or several levels. Several of the methods may be combined or the procedure may be performed using only the radiofrequency method of ablation.

A similar result may be obtained using a non-invasive ultrasound technique. In this alternative method, imaging may be performed using an ultrasound technique (or CT or MRI), and once the structures were localized, the ultrasound would be switched to high or low intensity focused ultrasound (HIFU or LIFU) and the paravertebral ganglia, alone or in combination with the rami, spinal nerve, anterior nerve root or DRG ablated. The frequency may be lowered, as desired, resulting in deeper penetration. Alternatively, the structures may be imaged using MRI and ablated using HIFU or LIFU. This method of ablation might be preferable to the minimally invasive method described above, since it does not involve skin penetration or pain.

A similar result may be obtained using another minimally invasive surgical technique. In this alternative method, a rigid or non-rigid endoscope with a camera and ablation tools such as stimulating wires, ultrasound, or any of the other methods already mentioned would be advanced through the intercostal space laterally to the paravertebral space for sympathectomy. An advantage of this method is that ganglia at several thoracic levels may be treated simultaneously.

A similar result may be obtained using electrical stimulation to inhibit sympathetic firing. The method may involve direct paravertebral access or indirect epidural access. Using this technique, firing patterns from autonomic fibres may be recorded by the stimulator and stimulation parameters altered accordingly.

The advantages of treating hypertension using either non-invasive or minimally invasive techniques described herein are numerous. Firstly, the para-vertebral ganglion cell is targeted rather than an axon. Cell bodies don't regenerate whereas axons may. Thus any reduction in blood pressure is likely to be permanent with this method. Furthermore, the density of the cell bodies in the ganglia is such that stimulating or ablating even a small area is likely to produce a much greater reduction in blood pressure than randomly ablating a small proportion of the nerve fibres surrounding a renal artery. Both techniques are simple and easy to perform. In the minimally invasive method, it involves a few needle insertions at different levels, each lasting a few minutes in an out-patient setting. It is a sequential approach which can be repeated later if necessary. The patient can be brought back for another procedure if the amount of blood pressure reduction is insufficient. This way, postural hypotension which results from excessive sympatholysis can be avoided. The non-invasive approach is even better, avoiding all discomfort to the patient. In comparison, renal nerve ablation is associated with several potential complications. Instrumenting the renal artery is not possible in up to 15% of patients in whom the procedure is attempted. Furthermore, instrumentation is associated with artery spasm or dissection and embolization into the substance of the kidney can cause further deterioration in renal function in kidneys already compromised by hypertension. Lastly, arterial punctures in the groin can be associated with groin hematoma or pseudo-aneurysm formation, as well as requiring groin compression.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method comprising reducing blood pressure of a patient by percutaneously accessing para-vertebral sympathetic ganglia, dorsal root ganglia or both; and irreversibly disabling said ganglia.

2. The method of claim 1 wherein said accessing comprises inserting an elongate member proximate or within the paravertebral sympathetic ganglia or dorsal root ganglia.

3. The method of claim 2 wherein said elongate member comprises a wire, a needle, or a catheter having a lumen therewithin.

4. The method of claim 3 further comprising inserting a camera through said catheter lumen.

5. The method of claim 1 wherein reducing blood pressure of a patient comprises permanently reducing the blood pressure of the patient.

6. The method of claim 1 wherein irreversibly disabling said ganglia comprises preventing regeneration of said ganglia.

7. The method of claim 1 further comprising denervating only a portion of the para-vertebral ganglia.

8. The method of claim 1 wherein irreversibly disabling said para-vertebral sympathetic ganglia, dorsal root ganglia or both comprises applying ablative means to said para-vertebral sympathetic ganglia, dorsal root ganglia or both.

9. The method of claim 8 wherein said ablative means comprises a chemical agent, mechanical means or electromagnetic energy selected from radiofrequency, microwave, ultrasound, high intensity focused ultrasound, low intensity focused ultrasound, infrared waves, electrical energy, laser energy, other sources of thermal energy including cooling, and combinations of the foregoing.

10. The method of claim 1 further comprising stimulating said para-vertebral sympathetic ganglia, dorsal root ganglia or both; monitoring a physiologic response related to said stimulating; applying ablative means to said para-vertebral sympathetic ganglia, dorsal root ganglia or both; and reducing said blood pressure.

11. The method of claim 1 wherein said para-vertebral ganglia or dorsal root ganglia is selected from any vertebral level between T6 and L1.

12. A method comprising reducing blood pressure of a patient by accessing a para-vertebral triangle; and irreversibly disabling neural structures therewithin.

13. The method of claim 12 wherein said neural structures are selected from sympathetic ganglia, dorsal root ganglia, grey or white rami, dorsal or ventral root, nerve fibers connecting said structures with a spinal cord, and combinations of the foregoing.

14. The method of claim 12 wherein said accessing comprises inserting an elongate member proximate or within the paravertebral sympathetic ganglia or dorsal root ganglia.

15. The method of claim 12 wherein said elongate member comprises a wire, a needle, or a catheter having a lumen therewithin.

16. The method of claim 12 further comprising inserting a camera through said elongate member.

17. The method of claim 12 wherein reducing blood pressure of a patient comprises permanently reducing the blood pressure of the patient.

18. The method of claim 12 wherein irreversibly disabling said ganglia comprises preventing regeneration of said ganglia.

19. The method of claim 12 further comprising denervating only a portion of the para-vertebral ganglia.

20. The method of claim 12 wherein irreversibly disabling said para-vertebral sympathetic ganglia, dorsal root ganglia or both comprises applying ablative means to said para-vertebral sympathetic ganglia, dorsal root ganglia or both.

21. The method of claim 20 wherein said ablative means comprises a chemical agent, mechanical means or electromagnetic energy selected from radiofrequency, microwave, ultrasound, high intensity focused ultrasound, low intensity focused ultrasound, infrared waves, electrical energy, laser energy, other sources of thermal energy including cooling, and combinations of the foregoing.

22. The method of claim 12 further comprising stimulating said para-vertebral triangle; monitoring a physiologic response related to said stimulating; applying ablative means to said para-vertebral triangle.

23. The method of claim 12 wherein said para-vertebral triangle is selected from any vertebral level between T6 and L1.

24. The method of claim 12 wherein said accessing comprises imaging said para-vertebral sympathetic ganglia, dorsal root ganglia or both prior to said disabling.

25. The method of claim 12 wherein said accessing comprises imaging said para-vertebral triangle prior to said disabling.

26. A method comprising treating heart failure, acute myocardial infarction, renal disease, or chronic renal failure by percutaneously accessing para-vertebral sympathetic ganglia, dorsal root ganglia or both; and irreversibly disabling said ganglia.

27. The method of claim 1 or 12, wherein said accessing is performed unilaterally.

28. The method of claim 1 or 12, wherein said accessing is performed bilaterally.

29. The method of claim 1 or 12, wherein said accessing is performed at one segmental location.

30. The method of claim 1 or 12, wherein said accessing is performed at multiple locations.

31. The method of claim 1 or 12, wherein said accessing is performed once.

32. The method of claim 1 or 12, wherein said accessing is performed several times.

33. A method comprising stimulating para-vertebral sympathetic ganglia, dorsal root ganglia or both of a patient; monitoring a physiologic response related to said stimulating; applying ablative means to said para-vertebral sympathetic ganglia, dorsal root ganglia or both; and reducing blood pressure of said patient.

34. A method comprising stimulating a para-vertebral triangle; monitoring a physiologic response related to said stimulating; applying ablative means to said para-vertebral triangle.

35. A device for reducing blood pressure, comprising an elongate tubular member with a proximal and distal end, adapted for percutaneous insertion proximate or within the para-vertebral sympathetic ganglia or dorsal root ganglia.

36. The device of claim 35 wherein a conductive wire is contained within the tubular member.

37. The device of claim 35 wherein a syringe is attached to the proximal end in fluid communication with the distal end.

38. The device of claim 37 wherein a neurolytic fluid is contained within the syringe.

39. The device of claim 35 wherein a camera is attached to the distal end.

40. The device of claim 36 wherein an alternating current energy source is electrically connected to the wire.

41. The device of claim 35 wherein an energy transducer is attached to the distal end.

42. The device of claim 35 wherein a mechanical ablation device is attached to the distal end.
